(19) United States
(12) Patent Application Publication
(10) Pub. No.: US 2013/0211312 A1
(43) Pub. Date: Aug. 15, 2013

(54) Prefilled Ocular Implants and Methods

(71) Applicant: Michael L. Gelvin, Alta Loma, CA (US)

(72) Inventor: Michael L. Gelvin, Alta Loma, CA (US)

(21) Appl. No.: 13/372,937

(22) Filed: Feb. 14, 2012

(51) Int. Cl. A61F 9/00

(52) U.S. Cl. 604/9

(57) ABSTRACT

A drainage device for implantation in an eye of a patient to treat an ocular condition includes a first tube configured to extend into an anterior chamber of the eye and convey aqueous humor from the anterior chamber. An exit portion is in fluid communication with the distal end portion. A passage extends between the distal portion of the first tube and the exit portion, with a liquid disposed in the passage. The implant may include a first seal associated with the distal end portion and a second seal associated with the exit portion to prevent drainage of the liquid through the exit portion. The implant may include an implant body disposed between the distal end portion and the exit portion, the passage extending through the implant body. The implant body may comprise a fluid-sensitive component in fluid communication with the passage that may be formed of a flexible membrane.

### BACKGROUND

The present disclosure relates generally to implants that are prefilled with liquid to maintain a suitable environment for liquid-sensitive components.

Glaucoma, a group of eye diseases affecting the retina and optic nerve, is one of the leading causes of blindness worldwide. Most forms of glaucoma result when the intraocular pressure (TOP) increases to pressures above normal for prolonged periods of time. TOP can increase due to high resistance to the drainage of the aqueous humor relative to its production. Left untreated, an elevated TOP causes irreversible damage to the optic nerve and retinal fibers resulting in a progressive, permanent loss of vision.

FIG. 1 is a diagram of the front portion of an eye that helps to explain the processes of glaucoma. In FIG. 1, representations of the lens 10, cornea 20, iris 30, ciliary body 40, trabecular meshwork 50, and Schlemm's canal 60 are pictured. Anatomically, the anterior segment of the eye includes the structures that cause elevated IOP which may lead to glaucoma. Aqueous humor fluid is produced by the ciliary body 40 which lies beneath the iris 30 and adjacent to the lens 10 in the anterior segment of the eye. This aqueous humor washes over the lens 10 and iris 30 and flows to the drainage system located in the angle of the anterior chamber. The angle of the anterior chamber, which extends circumferentially around the eye, contains structures that allow the aqueous humor to drain. The trabecular meshwork 50 is commonly implicated in glaucoma. The trabecular meshwork 50 extends circumferentially around the anterior chamber. The trabecular meshwork 50 seems to act as a filter, limiting the outflow of aqueous humor and providing a back pressure that directly relates to IOP. Schlemm's canal 60 is located beyond the trabecular meshwork 50. Schlemm's canal 60 is fluidically coupled to collector channels (not shown) allowing aqueous humor to flow out of the anterior chamber. The two arrows in the anterior segment of FIG. 1 show the flow of aqueous humor from the ciliary bodies 40, over the lens 10, over the iris 30, through the trabecular meshwork 50, and into Schlemm's canal 60 and its collector channels.

One method of treating glaucoma includes implanting a drainage device in a patient's eye. The drainage device allows fluid to flow from the interior chamber of the eye to a drainage site, relieving pressure in the eye and thus lowering IOP.

Some conventional drainage devices are primed prior to implantation. That is, during a surgical procedure, the surgeon introduces fluid into the drainage device in order to expel air and prepare the device for fluid flow. Because the devices are manufactured and stored dry, they are not conducive to being manufactured from materials that are liquid sensitive. Accordingly, the types of materials used in the devices are limited to materials able to withstand a dry shelf-life until they are implanted.

The system and methods disclosed herein overcome one or more of the deficiencies of the prior art.

### SUMMARY

In one exemplary aspect, the present disclosure is directed to a drainage device for implantation in an eye of a patient to treat an ocular condition. The implant may include a first tube having a distal portion and may be configured to extend into an anterior chamber of the eye and convey aqueous humor from the anterior chamber. The implant may also include an exit portion disposed in fluid communication with the distal end portion of the first tube. The exit portion may be disposed relative to the distal end portion to drain the aqueous humor to a drainage site in or around the eye. A passage may extend between the distal portion of the first tube and the exit portion, and a liquid may be disposed in the passage. The implant may include a first seal associated with the distal end portion disposed to prevent drainage of the liquid through the hollow lumen and a second seal associated with the exit portion disposed to prevent drainage of the liquid through the exit portion.

In one aspect, the first seal is one of a plug and a portion of the first tube. In another aspect, the implant includes an implant body disposed between the distal end portion and the exit portion, the passage extending through the implant body. The implant body may comprise a fluid-sensitive component that may be formed of a flexible membrane.

In another exemplary aspect, the present disclosure is directed to a drainage device for implantation in an eye of a patient to treat an ocular condition. The drainage device may include a plate portion disposable along a globe of the eye of the patient. The plate portion may include an inlet, an outlet, and a passage extending between the inlet and the outlet. The plate portion also may include a fluid sensitive component disposed adjacent to the passage. A drainage tube may extend from the inlet of the plate portion. The drainage tube may have a hollow lumen in fluid communication with the passage of the plate portion, the hollow lumen being sized to convey aqueous humor from the anterior chamber to the plate portion. A liquid may be disposed within the plate portion in communication with the fluid sensitive component. The drainage device may include a first seal cooperatively associated with the inlet of the plate portion, the first seal being configured to prevent the liquid from draining from the plate portion through the inlet. The drainage device also may include a second seal cooperatively associated with the outlet of the plate portion, the second seal being configured to prevent the liquid from draining from the plate portion through the outlet.

In yet another exemplary aspect, the present disclosure is directed to a method of manufacturing a drainage device for implantation in an eye of a patient to treat an ocular condition. The method may include steps of providing a plate portion sized and configured to be disposed along a globe of the eye of the patient, the plate portion comprising an inlet, an outlet, and a passage extending between the inlet and the outlet, the plate portion comprising a fluid sensitive component disposed adjacent the passage. A drainage tube may be associated with the inlet of the plate portion to place a hollow lumen of the drainage tube in fluid communication with the passage of the plate portion. The hollow lumen may be sized to convey aqueous humor from the anterior chamber to the plate portion. A liquid may be introduced into the plate portion in communication with the fluid sensitive component. The method may include cooperatively associating a first seal with the inlet of the plate portion to prevent the liquid from draining from the plate portion through the inlet, and cooperatively associating a second seal with the outlet of the plate portion to prevent the liquid from draining from the plate portion through the outlet.

In one aspect, cooperatively associating the first seal with the inlet includes sealing the drainage tube to prevent the liquid from draining through the drainage tube, and cooperatively associating the second seal with the outlet includes sealing a second tube associated with the outlet to prevent the liquid from draining through the drainage tube. In one aspect, cooperatively associating the first seal with the inlet includes introducing one of a crimp, a plug, a cap, and a dollop to the drainage tube. In one aspect, the first seal is a uniform part of the drainage tube, and associating a drainage tube with the inlet of the plate portion and cooperatively associating a first seal with the inlet of the plate portion are performed simultaneously.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram of the front portion of an eye.

FIG. 2 is a block diagram of an exemplary implant disposed in the eye in accordance with one embodiment of the present disclosure.

FIG. 3 is a schematic diagram of an exemplary ocular implant of FIG. 2 according to an exemplary aspect of the present disclosure.

FIG. 4 is a schematic diagram of a cross-sectional view of an exemplary ocular implant according to an exemplary aspect of the present disclosure.

FIGS. 5A-5G are schematic diagrams of exemplary distal end portions of the ocular implant according to an exemplary aspect of the present disclosure.

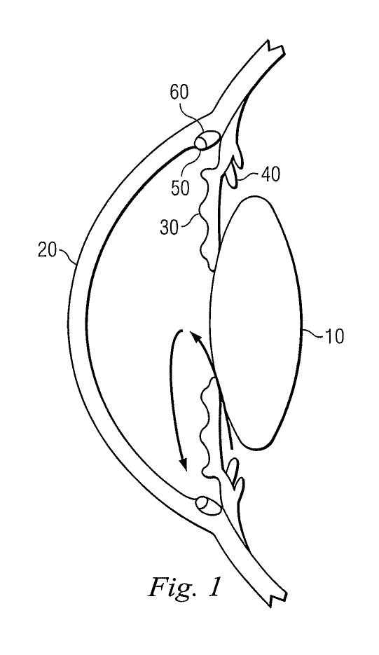

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure relates generally to a method and system for maintaining a fluid environment in an ocular implant prior to implantation in the eye. So doing simplifies the implantation process by permitting surgeons to implant the devices without requiring that a surgeon spend valuable time priming the implant with fluid prior to implementation. In addition, because the system maintains a wet environment, the ocular implant can be made of fluid sensitive components or materials. Fluid sensitive components or materials are components or materials that have properties that change when the component or material dries out. For example, some membranes in MEMS devices may include one or more chambers formed in part by flexible membranes that may be sensitive to prolonged exposure to dry air or a dry environment. As these membranes dry out, they may become less flexible, rendering them less responsive to pressure variations and less predictable after implantation. By maintaining a wet environment about these sensitive materials or components, the shelf life of the implant can be prolonged, and the overall system may be more robust than can be otherwise achieved.

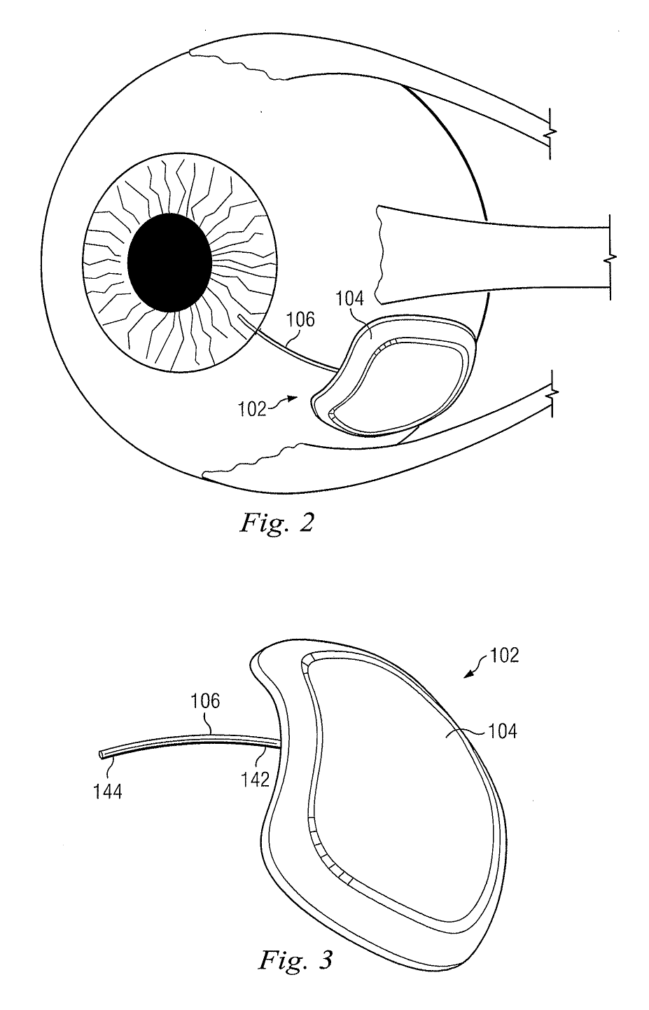

FIG. 2 shows an exemplary ocular implant 102 disposed on an eye according to one exemplary aspect of the present disclosure. The implant 102 includes a body referred to herein as a plate 104 and a drainage tube 106 that extends from the plate 104. The plate 104 is arranged to carry various components of an TOP control system, and may include a valve, pump, transducers or sensors, processing system and memory, drug delivery components, a power source or other components that may be used to either control the implant 102 or otherwise treat ocular conditions.

The plate 104 is configured to fit at least partially within the subconjunctival space and is sized for example within a range between about 15 mm×12 mm to about 30 mm×15 mm and has a thickness less than about 2 mm thick and preferably less than about 1 mm thick. The plate 104 may be formed to the radius of the eye globe (about 0.5 inches). It may be rigid and preformed with a curvature suitable to substantially conform to the globe or it may be flexible to conform to the globe. Some embodiments are small enough that conforming to the globe provides little benefit in comfort or implantation technique. The above dimensions are exemplary only, and other sizes and arrangements are contemplated.

When implanted, the plate 104 may be located in the subconjunctival pocket between the conjunctiva and sclera. It may be generally located on an ocular quadrant commonly used for conventional glaucoma drainage devices with plates; that is, it may be centered such that it is equidistant from the neighboring ocular muscles that define the ocular quadrant chosen for implantation. The drainage tube 106 is sized to bridge the anterior chamber and the plate 104 in the subconjunctival pocket to provide an auxiliary flow path for aqueous humor, bypassing the flow-resistive conventional pathway through the trabecular meshwork and shunting aqueous humor directly to a drainage site.

In the example shown, the drainage tube 106 is a single tube having a single lumen. Other embodiments include a plurality of drainage tubes or a plurality of lumens cooperating together to permit fluid to flow through the implant 102. The drainage tube 106 is sized to extend from the plate 104 to the anterior chamber of the eye, as shown in FIG. 2. Aqueous humor may drain through the drainage tube from the anterior chamber to and out of the plate 104 to alleviate elevated intraocular pressure conditions.

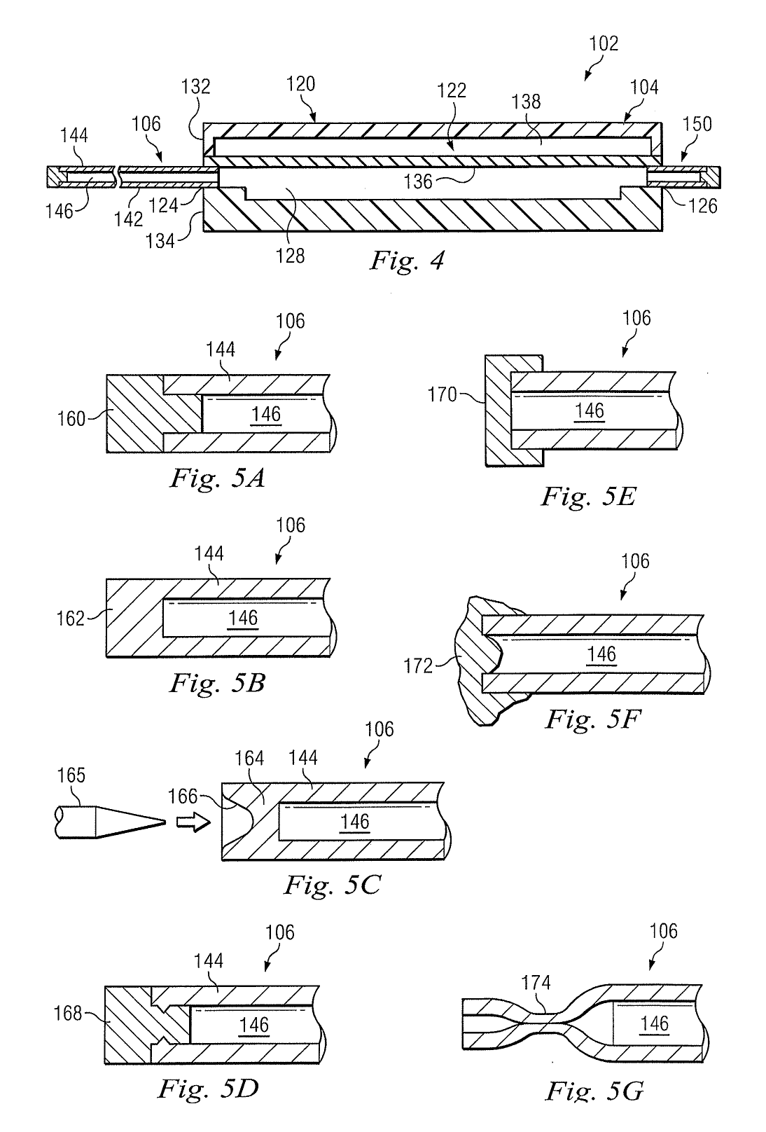

FIG. 3 shows the implant 102 independent of the eye and FIG. 4 shows a stylized cross-sectional view of the implant 102, as it may appear prior to implantation according to one embodiment. In this example, the implant 102 includes a housing 120 and a flow control portion 122 disposed in the housing 120. The housing 120 includes an inlet 124, an outlet 126, and a passage 128 between the inlet 124 and the outlet 126. In this example, the housing 120 includes an upper housing portion 132 and a lower housing portion 134. The flow control portion 122 may be used to regulate flow through the passage 128 from the inlet 124 to the outlet 126. In this embodiment, the flow control portion 122 includes a flexible membrane 136 between the upper and lower housing portions 132 and 134 respectively. Here, the passage 128 is formed between the lower housing portion 134 and the membrane 136. An actuation chamber 138 is disposed between the membrane 136 and the upper housing chamber 132. As a result of pressure fluctuations in the actuation chamber, the membrane 136 deflects toward or away from the lower housing portion 134 to increase and decrease the cross-sectional area of the passage 128. In so doing, the flexible membrane 136 increases and decreases flow resistance, enabling some level of control of the flow through the passage 128 and ultimately through the implant 102. The flow control portion 122 may include one or more valves, one or more pumps, one or more other flow control elements, or any combination of these.

The actuation chamber 138 may be used to create pressure variations that cause the flexible membrane 136 to deflect and increase and decrease the cross-sectional size of the passage 128. In one example, the actuation chamber 138 includes fluid and electrodes that change the fluid to gas to cause pressure variations. In other examples, the actuation chamber 138 is open to atmospheric pressure sources or other pressure sources that pressurize the chamber to cause the pressure variations.

The flexible membrane 136 may be formed of an elastically deformable elastomeric including without limitation, materials such as a silicone, silicon nitride, silicone elastomeric, polyimide, parylene and others. In the example shown, the flexible membrane is secured to the housing 120 at its edges. Although shown in cross section, the actuation chamber 138 may be disposed to form a circular or cylindrical chamber, with the flexible membrane 136 being secured along the diameter. Accordingly, the flexible membrane 136 may be a circular material secured at its periphery to the housing 120. As such, as the volume or pressure increases within the actuation chamber 138, the central portion of the flexible membrane 136 provides the highest level of displacement. In other embodiments, the housing 120 and flexible membrane 136 are formed so that the membrane has a non-circular shape, including oval, substantially rectangular, or square, for example. Other shapes are also contemplated.

Because of its material or sensitive nature, some membranes have a relatively short shelf-life. They may dry out after being assembled into the implant 102, while waiting to be used. As the membranes dry, they may frequently become less flexible, rendering them less responsive to pressure variations and less predictable after implantation. However maintaining the flexible membranes in a wet environment may alleviate these challenges. Here, the passage 128 may be filled with a fluid or liquid that maintains the wet environment, prolonging the life of the membrane. In one embodiment, the fluid is a saline solution. In one example, the fluid is Alcon BSS PLUS®, sold by the assignee of the present disclosure.

Still referring to FIG. 4, the drainage tube 106 includes a proximal end portion 142, a distal end portion 144, and a hollow lumen 146 extending therebetween. The proximal end 142 connects the hollow lumen 146 to the passage 128 through the plate 104. The distal end portion 144, however, includes a seal that cooperates with the lumen 146 and may be used to prevent the liquid from exiting the implant 102 through the drainage tube 106. As described in greater detail below, the seal may be formed of a plug, cap, crimp, other element, or may be formed of a portion of the material of the drainage tube 106.

In the example in FIG. 4, in addition to the drainage tube 106 extending from the inlet 124 of the plate 104, the ocular implant 102 includes an exit portion 150, shown as a second drainage tube that is intended to release fluid to a drainage site after passing through the plate 104 and out of the outlet 126. Similar to the distal end portion 144 of the drainage tube 106, the exit portion 150 also includes a seal. While shown in FIG. 4 as a secondary drainage tube, the exit portion 150 may be nothing more than the outlet 126 from the implant plate 104, so long as the outlet may be sealed to prevent the liquid from draining from the implant 102.

With both the distal end portion 144 and the exit portion 150 of the implant 102 sealed closed, the liquid may be prevented from draining from the implant 102 during storage. Therefore, implants that have fluid-sensitive components may tend to not dry out, thereby increasing the shelf-life of the implant. In addition, since the implant can be filled with a fluid, such as a saline, the implant need not be separately primed prior to use, making the implantation surgical procedure simpler and more efficient.

FIGS. 5A-5G show enlarged cross-sectional views of exemplary distal portions 144 of the drainage tube 106 of the implant 102 to seal the passage 128 and prevent fluid drainage until the seal is removed. For ease of explanation, the exit portion 150 is not shown or described further with the understanding that the same seals used with the distal portions 144 may be used with the exit portion 150. In one embodiment, the same type of seal used on the entrance to the drainage tube 106 is also used on the exit portion 150. However, it should be understood that different types of seals may be used at each end of the implant 102.

FIG. 5A shows one embodiment of a seal 160 of the drainage tube 106 that prevents fluid from prematurely exiting the implant 102. Here the seal 160 is a plug pressed into the lumen 146 of the drainage tube 106. In use, the plug 160 may be removed from the drainage tube by simply pulling it from the tube. Alternatively, a surgeon may cut or snip off the end of the drainage tube, including the plug, which may be discarded. A surgeon may remove the plug just prior to implanting the ocular implant into the eye. Since the plug is intended to maintain a particular fluid level within the ocular implant 102, and since the ocular implant will already be fluid-filled, there is no need to prime the implant, and any fluid-sensitive components can be maintained in operable condition.

The plug 160 may be formed of a biocompatible material capable of cooperating with the drainage tube to seal and prevent fluid loss from the implant 102. For example, the plug may be formed of any suitable biocompatible material including metals such as cobalt-chromium alloys, titanium alloys, nickel titanium alloys, and/or stainless steel alloys. Ceramic materials such as aluminum oxide or alumina, zirconium oxide or zirconia, compact of particulate diamond, and/or pyrolytic carbon may be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Biocompatible cements and adhesives also may be used to form the plug 160. In one example, the plug 160 is formed of the same type of material as the drainage tube 106.

FIG. 5B shows an embodiment of a seal 162 formed as an integral part of the drainage tube. Accordingly, in this embodiment, the seal 162 may be formed during tube manufacturing, and the tube and the seal together form a monolithic or single, uniform element. The seal may be removed by snipping the end of the drainage tube or by perforating the end of the drainage tube. FIG. 5C shows a seal 164 formed as a part of the drainage tube 106 having a concavity 166 in the end. The concavity, in this example, is configured to assist with receiving a puncture device 165, such as a needle shown in FIG. 5C. The puncture device may be used to penetrate the seal to the lumen 146 to create a drainage opening in the distal portion of the drainage tube 106. FIG. 5D shows a seal 168 formed of a plug having a single radial thread. The plug may be popped out or twisted out of the drainage tube 106 to open the lumen 146. Any number of threads may be used. In one embodiment, the plug and tube have one or more spiral threads allowing the plug to be unscrewed or unthreaded from the drainage tube 106. In another embodiment, the plug is not threaded but has nubs or projections on one of the plug and the tube inner diameter that interfaces and provides interference from removal. FIG. 5E shows a seal 170 as a cap that is placed on or adhered to the drainage tube 106. In the example shown, the cap is adhered to the tube 106 with a biocompatible adhesive. However, in other embodiments, the cap may be threaded onto the tube or may fit onto the end of the tube with a friction fit. The cap may be removed or may be snipped off to permit fluid flow. FIG. 5F shows one embodiment with a seal 172 formed of a biocompatible cement or adhesive. In this embodiment, the seal 172 is a dollop disposed over the end of the drainage tube 106 in a manner to seal the drainage tube. FIG. 5G shows one embodiment where the seal 174 is a crimped portion in the drainage tube 106. In one embodiment, the crimped portion is made using a heated pinching tool that may thermoset the distal portion of the tube. The crimped portion may be snipped off to enable flow. These seals are simply examples that allow the ocular implant 102 to be filled with fluid and maintain the fluid within the implant. Other seals are also contemplated.

In any of the examples described above, the seal may be formed of a bioresorbable material. In one example, the seal is a plug formed of a bioresorbable material that initially prevents drainage flow of aqueous humor through the implanted ocular implant. However, over time, the bioresorbable material dissolves, thereby opening a pathway for fluid to flow into and through the drainage tube. Examples of suitable bioresorbable materials include, along others, polylactide, polyglycolide, tyrosine-derived polycarbonate, polyanhydride, polyorthoester, polyphosphazene, calcium phosphate, hydroxyapatite, bioactive glass, and combinations thereof. Bioresorbable materials may permit a surgeon to implant the ocular implant without first removing the seals. Since the bioresorbable material must degrade before flow can occur, the drainage functionality is not enabled for a period of time after the surgery is complete. This may allow the body to begin its healing process prior to drainage occurring. The length of delay may be controlled by the type and thickness of the bioresorbable material used. In different embodiments, the delay between implantation may be at least one day, at least three days, at least a week, at least three weeks, or at least a month. Shorter and longer times are also contemplated.

In use, the ocular implant 102 may be filled with a fluid during the manufacturing process. Since the sealed ends of the implant prevent drainage and evaporation, the fluid may be maintained within the ocular implant during the time that the implant is stored for use. Therefore, the implant may be manufactured with fluidically sensitive materials, such as the flexible membrane discussed above, without concern for the material drying out or changing properties. This increases the overall shelf life of the ocular implant. Furthermore, in embodiments where the fluid is biocompatible, the implant is pre-primed. This can save surgical time because the surgeon is not required to flush the implant to remove all air that might otherwise be entrapped in the implant.

Accordingly, one exemplary method of manufacturing includes assembling an ocular implant with a fluid sensitive component or material. This may include assembling or otherwise providing the plate 104 with a flow control portion disposed therein. The flow control portion may, depending on the embodiment, comprise one or more of valve or pump portion that includes the fluid sensitive component. In one embodiment, the fluid sensitive component is a flexible membrane. The device may be assembled by attaching upper and lower housing portions to capture the fluid sensitive component therebetween, forming a part of a passage in the plate 104. With the plate 104 assembled, the drainage tube 106 may be introduced into the inlet of the plate 104 so that the lumen of the drainage tube is in communication with the passage through the plate 104. Some embodiments include a second drainage tube introduced into the outlet of the plate. A liquid may be used to flush the implant to remove all air entrapped in the implant. With the fluid introduced into the ocular implant, the entrance and exits may be sealed using any of the seals or methods discussed above. For example, in one embodiment, sealing the ocular implant occurs using a plug introduced into the drainage tube 106. In another exemplary embodiment, sealing occurs using crimping or other deformation of a portion of the drainage tube. In other examples, the drainage tube itself is formed with sealed ends. Alternatively, the ends of the implant may be dipped into an adhesive. It should be noted that in some examples, attaching the drainage tube simultaneously seals the inlet. Sealing one port of the implant may be performed before filling the tube. In other embodiments, the implant includes a separate filling port.

With the ends of the ocular implant 102 sealed, the implant may be packaged for use. In use, a surgeon may remove the ocular implant 102 from its packaging. In one example, the surgeon may use a pair of surgical scissors to snip off the sealed ends of the implant prior to introducing the device into the eye. In another example, the end of the implant is punctured to provide drainage access. In yet another example, a plug or cap is pulled, dissolved, or otherwise removed.

In one example, the surgeon may implant the ocular implant into a patient's eye without removing the seal. Then at a later point in time, after the implantation surgery is complete, the surgeon may perform a revision procedure that may include removing the seal from the ends of the implant to permit drainage flow through the implant. The revision surgery may include accessing and removing a plug or cap, snipping off the sealed portion, or puncturing the tube to create a flow port in the implant with a sharp tool, such as a needle.

In embodiments employing bioresorbable materials, the implant may be implanted during a surgical procedure without removing the seals. Later, after the surgery is complete, the bioresorbable material may sufficiently erode or be resorbed until fluid flow may occur through the implant. In one example, this may occur at least a day after surgery, at least three days, at least a week, at least three weeks, or at least a month after surgery. Shorter and longer times are also contemplated.

It should be noted that both the drainage tube and the exit at the drainage site may be treated in the same manner to permit or prevent fluid flow using any of the devices of methods in the exemplary embodiments above.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure

1. A drainage device for implantation in an eye of a patient to treat an ocular condition, the drainage device comprising:
a drainage first tube having a distal end portion and being configured to extend into an anterior chamber of the eye and convey aqueous humor from the anterior chamber;
an exit portion disposed in fluid communication with the distal end portion of the drainage tube, the exit portion being disposed relative to the distal end portion to drain the aqueous humor to a drainage site in or around the eye;
a passage extending between the distal portion of the drainage tube and the exit portion;
an implant body disposed between the distal end portion and the exit portion, the passage extending through the implant body, the implant body comprising a fluid sensitive component;
a liquid disposed in the passage, the liquid in contact with the fluid sensitive component to preserve the fluid sensitive component;
a first seal associated with the distal end portion disposed to prevent drainage of the liquid through the hollow lumen; and
a second seal associated with the exit portion disposed to prevent drainage of the liquid through the exit portion.

2. The drainage device of claim 1, wherein the first seal is one of a plug and a portion of the drainage tube.

3. (canceled)

4. (canceled)

5. The drainage device of claim 1, wherein the fluid-sensitive component is a flexible membrane.

6. The drainage device of claim 5, wherein the flexible membrane is part of at least one of a valve and a pump.

7. The drainage device of claim 1, wherein the exit portion is an outlet disposed in the implant body.

8. The drainage device of claim 1, further comprising a second tube, wherein the exit portion is a part of a second tube.

9. The drainage device of claim 1, wherein at least one of the first and second seals are formed of a bioresorbable material.

10. A drainage device for implantation in an eye of a patient to treat an ocular condition, the drainage device comprising:
a plate portion disposable along a globe of the eye of the patient, the plate portion comprising an inlet, an outlet, and a passage extending between the inlet and the outlet, the plate portion comprising a fluid sensitive component disposed adjacent the passage;
a drainage tube extending from the inlet of the plate portion, the drainage tube having a hollow lumen in fluid communication with the passage of the plate portion, the hollow lumen being sized to convey aqueous humor from the anterior chamber to the plate portion;
a liquid disposed within the plate portion in communication with the fluid sensitive component the liquid preserving the fluid sensitive component;
a first seal cooperatively associated with the inlet of the plate portion, the first seal being configured to prevent the liquid from draining from the plate portion through the inlet; and
a second seal cooperatively associated with the outlet of the plate portion, the second seal being configured to prevent the liquid from draining from the plate portion through the outlet.

11. The drainage device of claim 10, wherein the first seal is disposed at a distal end portion of the drainage tube.

12. The drainage device of claim 10, comprising a second tube extending from the outlet of the plate portion, wherein the second seal is disposed at an end portion of the second tube.

13. The drainage device of claim 10, wherein the second seal is disposed over the outlet in the plate.

14. The drainage device of claim 10, wherein the first seal is one of a crimp, a plug, a cap, or and a dollop.

15. The drainage device of claim 10, wherein the first seal is a single, uniform element of the drainage tube.

16. The drainage device of claim 10, wherein the fluid sensitive component comprises a flexible membrane.

17. The drainage device of claim 16, wherein the flexible membrane is a part of at least one of a valve and a pump.

18. A method of manufacturing a drainage device for implantation in an eye of a patient to treat an ocular condition, the method comprising:
providing a plate portion sized and configured to be disposed along a globe of the eye of the patient, the plate portion comprising an inlet, an outlet, and a passage extending between the inlet and the outlet, the plate portion comprising a fluid sensitive component disposed adjacent to the passage;
associating a drainage tube with the inlet, of the plate portion to place a hollow lumen of the drainage tube in fluid communication with the passage of the plate portion, the hollow lumen being sized to convey aqueous humor from the anterior chamber to the plate portion;
introducing a liquid into the plate portion in communication with the fluid sensitive component;
preserving the fluid sensitive component with the liquid;
cooperatively associating a first seal with the inlet of the plate portion to prevent the liquid from draining from the plate portion through the inlet; and
cooperatively associating a second seal with the outlet of the plate portion to prevent the liquid from draining from the plate portion through the outlet.

19. The method of claim 18, wherein cooperatively associating the first seal with the inlet includes sealing the drainage tube to prevent the liquid from draining through the drainage tube, and wherein cooperatively associating the second seal with the outlet includes sealing a second tube associated with the outlet to prevent the liquid from draining through the drainage tube.

20. The method of claim 18, wherein cooperatively associating the first seal with the inlet includes introducing one of a crimp, a plug, a cap, or and a dollop to the drainage tube.

21. The method of claim 18, wherein the first seal is a uniform part of the drainage tube, and wherein associating a drainage tube with the inlet of the plate portion and cooperatively associating a first seal with the inlet of the plate portion are performed simultaneously.
